(19) United States
(12) Patent Application Publication
(10) Pub. No.: US 2026/0157985 A1
(43) Pub. Date: Jun. 11, 2026

(54) METHOD TO TREAT SYMPTOMS ASSOCIATED WITH ACNE, ECZEMA, PSORIASIS, AND RELATED DERMATOLOGICAL CONDITIONS WITH COMPOSITION COMPRISING A PROANTHOCYANIDIN, SPHINGOLIPID, AND SULPHUR COMPOUNDS

(71) Applicant: Clark L. Swanson, Henderson, NV (US)

(72) Inventor: Clark L. Swanson, Henderson, NV (US)

(21) Appl. No.: 19/041,016

(22) Filed: Jan. 30, 2025

Related U.S. Application Data
(60) Provisional application No. 63/626,678, filed on Jan. 30, 2024.

(51) Int. Cl. A61K 31/164; A61K 9/00; A61K 31/10; A61K 31/353; A61K 33/04; A61K 36/87; A61K 38/48; A61K 47/12; A61K 47/38

(52) U.S. Cl. CPC A61K 31/164; A61K 9/0053; A61K 31/10; A61K 31/353; A61K 33/04; A61K 36/87; A61K 38/4873; A61K 47/12; A61K 47/38

(57) ABSTRACT

Use of sphingolipids as an active ingredient of a stimulator as a source of proanthocyanidins and a source of sulfur in the treatment of acne, eczema, psoriasis or related dermatological conditions. The active ingredient reduces transepidermal water loss (“TEWL”) and stimulates the natural moisture factor (“NMF”), which acts as a catalyst for improved synthesis of antibacterial activity from Methylsulfonylmethane's (“MSM”) contributed sulphur. The reduction of TEWL in turn causes an increase in available transepidermal hydration which leads to an increase in penetrability by the antibacterial agent, namely a sulfur (“MSM”) and precipitates the potentiation of a proanthocyanadin to modulate the inflammatory pathways, such as nuclear factor-kappa B (NF-κB), and thereby altering the environment of molecular bacterial adhesion and penetrability of the stratum cornea for the delivery of anti-bacterial and anti-inflammatory agents.

### CROSS-REFERENCE TO RELATED APPLICATION(S)

The present application derives priority from U.S. Provisional Patent Application 63/626,678 filed 30 Jan. 2024.

### BACKGROUND OF THE INVENTION

### Field of the Invention

The invention relates to the use of sphingolipids to reduce transepidermal water loss, which increases Natural Moisuture Factor (“NMF”) increasing hydration which allows for the improvement of the microbiota environment, increasing penetrability and efficacy of a stimulator comprised of antibacterial sulfur and a proanthocyanadin which modulates the inflammatory pathways. Such is advantageous to relieving symptoms of acne, psoriasis, eczema, and associated dermatological conditions.

### Description of the Background

The pathogenesis and progression of skin disease is underpinned by oxidative stress and involves the introduction of free radical components such as reactive oxygen species (ROS), reactive nitrogen species (RNS) or lipid peroxidation (LPO). Antioxidant-related therapies such as the use of nutraceuticals are of particular growing interest in the segment of the global population with skin conditions. While the topical application of medicine and other therapeutic compounds have been attractive for the administration of drugs, the ability to deeply penetrate the skin's multiple layers has underperformed and few have provided proven scientific or conclusive results. As such, development of certain topical applications have proven to be promising, such as hydrogels, nanofibers, nanoparticles, and natural products. (Antibiotic Delivery Strategies to Treat Skin Infections When Innate Antimicrobial Defense Fails, Antibiotics (Basel). 2020 February; 9(2): 56., Pubmed: PMCID: PMC7168299, PMID: 32024064).

“Traditionally, the NF-κB pathway is thought of as a pro-inflammatory signaling pathway responsible for the upregulation of genes encoding cytokines, chemokines, and adhesion molecules [89]. The inhibitory effect of MSM on NF-κB results in the downregulation of mRNA for interleukin (IL)-1, IL-6, and tumor necrosis factor-α (TNF-α) in vitro [90,91]. As expected, translational expression of these cytokines is also reduced; furthermore, IL-1 and TNF-α are inhibited in a dose-dependent manner [90].” (Methylsulfonylmethane: Applications and Safety of a Novel Dietary Supplement, Nutrients. 2017 March; 9(3): 290. Published online 2017 Mar. 16. doi: 10.3390/nu9030290, PMCID: PMC5372953, PMID: 28300758).

The anti-inflammatory and anti-bacterial activity of proanthocyanidins however is not the scope of the present invention and the existing prior art does therefore not apply in this context, proanthocyanidins may influence other enzymes and pathways involved in inflammation, including lipoxygenase (LOX) and nitric oxide synthase (NOS). These compounds exhibit antioxidant properties and may scavenge free radicals, further contributing to their anti-inflammatory and protective effects.

Skin disease is highly prevalent in the United States (U.S.) affecting about 60% of the population at some point in their lifetime. The prevalence of skin disease requiring medical intervention is about 20%, and consequently, about 10% of a primary care physician's workload and 6% of hospital outpatient referrals are attributable to them. Among all skin disease cases about 17 million are attributable to acne that may present at various stages of severity, from mild or occasional break-outs to the severe inflammation and infection of acne vulgaris associated with gram negative folliculitis. The most common dermatoses after acne are atopic dermatitis (eczema) and psoriasis. The following summarizes each of these skin diseases.

### Acne

### Epidemiology

Acne vulgaris is prevalent worldwide and has seen an increasing incidence, particularly among adolescents aged 15-19. In the United States (U.S.), approximately 17 million individuals are affected by acne, with nearly 85% of those aged 12-24 experiencing the condition. This makes acne the most widespread skin disease in the country. The ongoing rise in acne prevalence indicates a significant, unaddressed demand for dermatologic care and highlights potential opportunities for enhancing accessibility and delivery of such care. Acne, affecting up to 17 million individuals, stands as the most prevalent skin condition in the United States. It is particularly common during childhood, adolescence, and young adulthood, often characterized by a cycle of outbreaks and remissions. In some cases, it may manifest as the serious and chronic condition known as Acne Vulgaris. Additionally, environmental contaminants and psychological stress can serve as triggers for acne outbreaks.

### Pathophysiology

Acne, a form of folliculitis, is marked by inflammation of hair follicles and an underlying infection, often bacterial or fungal. Its onset typically occurs during puberty, coinciding with the rapid increase in the body's production of hormones like dihydrotestosterone or testosterone. This condition is specifically characterized by the presence of pro-inflammatory elements that disrupt the integrity of the dermal hair follicular walls within the skin's epidermal layer. Secondary infections commonly result from anaerobic, gram-positive bacteria, notably Propionibacterium acnes (P. acnes), and are also attributed to the Malassezia fungus, which has a strong association with seborrheic dermatitis. Propionibacterium acnes (P. acnes) is a typically present gram-negative, anaerobic bacteria on the skin's surface. Despite its anaerobic nature, it thrives in aerobic environments, such as those found on skin surfaces. Hormonal fluctuations, particularly during puberty, lead to an increase in sebaceous glands' production of lipids (sebum), resulting in elevated lipid secretion into hair follicles and making the skin oilier. The migration of P. acnes bacteria in lipid-rich environments leads to the overcolonization of hair follicles. This overcolonization manifests as the development and distribution of biofilm colonies, varying in degrees within the hair follicle. The overcolonization of P. acnes bacteria is compounded by the sebaceous plugging of hair follicles at the skin surface. This creates an anaerobic, less oxygenated environment that is conducive to the overcolonization of P. acnes bacteria. The occlusion of follicles leads to the formation of a nidus of infection, fostering the development of pro-inflammatory environments associated with immune dysregulation. This, in turn, triggers the release of reactive chemical species, including reactive oxygen species (ROS) and reactive nitrogen species (RNS), promoting oxidative stress. The resulting oxidative stress initiates inflammatory responses in a cyclic pattern of events, further perpetuating oxidative stress.

### Treatment

Traditionally, acne has been addressed through the use of prescription medications, including benzoyl peroxide, tretinoin (retinoic acid), and antibiotics such as clindamycin, doxycycline, erythromycin, and sulfur. However, with the exception of sulfur compounds, antibiotics are deemed unsuitable for prolonged use due to their potential to suppress the normal bacterial flora of the intestines. This suppression can lead to the overcolonization of other bacterial species, some of which may develop resistance to the antibiotics. The extended use of antibiotics poses risks, including interference with normal colonic functioning, such as nutrient absorption, and may give rise to abdominal infections that are potentially life-threatening. Despite these drawbacks, dermatological treatment has traditionally relied on antibiotics for acne management. Contraceptives have demonstrated efficacy in improving acne for some women. However, the use of oral contraception is limited to the female population and is more limited to hormonal related outbreaks. In more severe cases of acne, oral isotretinoin (Accutane) has been employed. While oral isotretinoin suppresses sebaceous gland activity, indirectly reducing inflammatory lesions, it is not a curative drug. Discontinuation of treatment often leads to a recurrence of acne, necessitating appropriate maintenance treatment. Despite potential side effects associated with all acne medications, isotretinoin is particularly toxic, causing birth defects and liver disease, making it a last resort. Although Accutane is no longer available in the U.S., its generic counterpart, isotretinoin, is accessible but more rarely used. Consequently, there is a demand among acne patients for an alternative, safer, and long-term effective treatment.

### Eczema

### Epidemiology

Globally, nearly 20 percent of children and 3 percent of adults suffer from some form of eczema. Recognized risk factors for developing this dermatosis include residing in developed nations or colder climates. In the U.S., an estimated 32 million people exhibit symptoms of eczema, with 18 million experiencing atopic dermatitis. Atopic dermatitis, a form of eczema, is prevalent in individuals with a genetic predisposition and families with allergic conditions, such as asthma and allergic rhinitis. Its highest prevalence is observed among children, with nearly two-thirds reporting a food allergy. A significant and distinct clinical manifestation of eczema, specifically hand dermatitis, constitutes 20-35% of all U.S. eczema cases, impacting approximately 10% of the U.S. adult population. In a 2007 survey involving individuals in the U.S. exhibiting symptoms of eczema, only 37% of respondents reported receiving a physician's diagnosis. Seventy percent of eczema cases initiate in children younger than 5 years old, with around 60% of infants with eczema continuing to experience one or more symptoms into adulthood.

### Pathophysiology

Atopic dermatitis, the prevalent form of eczema, is defined by three key conditions: genetic predisposition, a family history of allergic conditions (e.g., asthma or allergic rhinitis), and the compromise of the integrity of the epidermal skin barrier. Clinically, it is marked by the fluctuation between exacerbation and remission of patches exhibiting dry, scaly, rough, discolored, and inflamed skin areas. Eczema is susceptible to exacerbation by environmental factors, such as irritants and pollution, along with emotional and psychological triggers. Eczema is a chronic condition characterized by frequent exacerbations that may go into remission but can recur at any time. Clinically, it presents as small raised exudative lesions around the flexor areas of the arms and legs that are fluid-filled and exudative when scratched and itchy. This pruritus heightens the risk of infection, leading to the development of patches of dermatitis that become thickened and discolored. Moreover, it often manifests in the skin around the head of children, a condition commonly referred to as ‘cradle cap.’ Atopic dermatitis affects between 8.7%-18.1% of all infants and young children.

### Treatment

Currently, eczema is commonly treated with antihistamines for itching, such as diphenhydramine, topical and/or oral corticosteroids like betamethasone and prednisone, calcineurin inhibitors such as tacrolimus and pimecrolimus to address immune-related manifestations, and antibiotics such as penicillin and penicillin-like drugs. Antibiotics have been a staple in eczema treatment for decades. This treatment is ex-post and is meant to address the infectious consequences of eczema but does not target its root cause, such as its association with an overactive response by the body's immune system. To address severe outbreaks of eczema, oral steroids may be employed, but they come with the risk of side effects, including immune suppression, elevated blood sugar, and blood pressure. The mechanism of action involves repressing abnormally functioning T cells and macrophages, cytokines such as NL-kB, IL-4, IL-5, immunoglobulin IgE, and B cells and eosinophils-types of white blood cells directly involved in allergic reactions. These chemical moieties and cells are overproduced due to dysregulation of the body's immune system (Ohmen J D et al., J Immunol., 154: 1956-1963, 1995—Overproduction of IL-10; Hamid Q et al., J Clin. Invest., 94: 870-876, 1994—Cytokine expression; which are incorporated by reference herein in their entireties). Steroid therapy is not recommended for children who suffer from such skin conditions due to observed side effects like immunosuppression, inhibited growth, and adrenal fatigue.

### Psoriais

### Epidemiology

Estimates of the prevalence of psoriasis have varied across studies. Systematic reviews of international population-based studies on the global epidemiology of psoriasis revealed wide variation. The prevalence of psoriasis in adults ranged from 0.91 to 8.5 percent, and in children, it ranged from 0 to 2.1 percent. The occurrence of psoriasis varies by age and geographic region, becoming more prevalent in nations farther from the equator. Prevalence estimates also varied in relation to demographic characteristics, with studies confined to adults reporting higher estimates of psoriasis compared to those including all age groups. Importantly, the incidence (number of new cases per 1000 persons in the population per year) and the prevalence of this disease (the total number of cases in the population at any given time) have risen for decades.

### Pathophysiology

The pathological underpinning of psoriasis is the targeting and harm of a person's skin cells by their own immune system. Diagnostic differentiation of psoriasis types is typically based on the clinical evaluation of a patient's signs and symptoms. Psoriasis is a chronic autoimmune skin disease characterized by patches of abnormal skin that are red, dry, itchy, and scaly. These psoriatic skin changes may be triggered in areas of the skin subjected to environmental irritant factors. Psoriasis is generally believed to have a genetic basis. The observation that an identical twin has a three-fold risk of developing psoriasis if the other twin has the condition, as opposed to a significantly lesser risk for a non-identical twin, gives credence to this condition having a genetic basis.

Common signs and symptoms comprise red hyperkeratotic patches of skin covered with thick, silvery scales, small scaling spots (often observed in children), dry and cracked skin prone to bleeding, accompanied by itching, burning, or soreness. Additionally, there may be thickened, pitted, or ridged nails, and the condition may be linked with swollen and stiff joints (psoriatic arthritis). Psoriasis typically follows cycles, with flare-ups lasting for a few weeks or months, followed by periods of subsiding or even complete remission. Psoriasis manifests in five main types: plaque, guttate, inverse, pustular, and erythrodermic. Plaque psoriasis, or psoriasis vulgaris, constitutes approximately 90% of cases, characterized by red patches covered with white scales. Symptoms may intensify in winter and during treatment with medications like beta-blockers (commonly used for hypertension) and non-steroidal anti-inflammatory drugs (NSAIDs) such as ibuprofen and naproxen used for pain relief. Triggers for exacerbating psoriasis include skin infections and psychological stress.

### Treatment

Psoriasis lacks a cure; hence, treatments focus on symptom reduction, addressing pain, inflammation, and scaling. Treatment approaches often involve a combination of topical creams, oral or injected medications, and light therapy. Psoralens, a drug class, is employed alongside light therapy. Oral drugs like methotrexate and biologics such as the monoclonal antibodies ustekinumab and secukinumbab are utilized to suppress the immune system, reducing inflammation in individuals with psoriasis.

It is noteworthy that medications employed to alleviate inflammation in acne, such as retinoids, and those used to diminish inflammation and hyperkeratosis in eczema, like tacrolimus and pimecrolimus, as well as light therapy, find application in the treatment of psoriasis. What is lacking is a treatment approach that addresses the fundamental molecular and histological irregularities causing these dermatoses, aiming to enhance the human condition. This would involve averting disruptions to the body's homeostasis, such as the intestinal bacterial flora, or the suppression of the immune system in individuals with the aforementioned dermatoses using drugs that have the potential for adverse drug events, thereby creating disease states through their unwanted side effects.

### SUMMARY OF THE INVENTION

It is, therefore, an object of the present invention to provide a composition for use in dermatological application which is effective for treating skin disorders over long term use without the side effects of steroids, antibiotics, anti-fungal, and other treatments. The need for treating these skin conditions also relates to the delivery of medicine at the point of insult-topical applications of creams, lotions, steroids, or other modalities have not proven effective long term. Antibacterial resistance is also a factor when considering the long term use of traditional interventions. While there is supporting evidence for the use of certain compounds that have shown benefit for acne, psoriasis, and eczema, or associated conditions, there has not been a system methodology identified to take into account density of the particles such as biologically produced sulfur particles with hydrophilic properties to combine with a natural moisture factor that has particular affinity for through the stratum corneum layer. The system and its use in no way prohibits others from using the natural products contained within the parameters of the system in other way. However the system specificity of using natural products in the manner and in the amounts specifically described are the basis for the method that underlies the system.

According to the present invention, the above-described and other objects are accomplished by providing an orally-administered composition comprising one or more ceramides, an 18-carbon amino alcohol with an unsaturated hydrocarbon chain, together with a sulfur compound, such as methyfulfonylmethane (MSM), and a proanthocyanadin, preferably derived from grape seed extract, for the prevention and treatment of acne, eczema, psoriasis and other skin conditions, with an adjuvant enzyme preferably from bromelain.

The ceramide is preferably a phytosphingosine (2-amino-4-octadecene-1,3-diol), and most preferably rice-derived phytosphingosine, although a variety of other choices for phytoceramide can be made, such as those derived from potatoes or wheat, and the sulfur compound is preferably MSM, although a variety of other choices for sulfur compounds and be made. The ceramide is combined with a sulfur compound.

The proanthocyanadin is preferably derived from grape seed extract. Proanthocyanidins are a group of flavonoids found in the non-volatile parts of plants, particularly in seeds, bark, and skin. Contrary to essential oils, which consist of volatile aromatic compounds, proanthocyanidins are known for their presence in these non-volatile plant tissues. The chemical structure of proanthocyanidins involves cyclic molecules with more complex formulas, typically composed of C15H14O6 or similar structures. Proanthocyanidins, also known as condensed tannins, comprise oligomers and polymers of monomeric flavans linked through specific single (B linkages) and double (A linkages) bonds. Despite not being terpenes, proanthocyanidins exhibit substantial antioxidant activity, contributing to their significance as secondary plant metabolites. Proanthocyanidins can also be extracted from French tree bark, jubjube berry, apples, cranberries, pecans, pistachios, and a wide variety of other sources.

The composition may also comprise one or more adjuvants and one or more excipients for oral administration including Cellulose Gel, Calcium Carbonate, or Stearic Acid. In the preferred embodiment Microcrystalline Cellulose, Silicon Dioxide, Vegetable Magnesium Stearate is used.

The adjuvant is preferably an enzyme, and most preferably bromalaise derived from bromelain. Enzymes that are involved in processes like exfoliation, anti-inflammatory responses, and sebum regulation.

In another aspect, provided herein is a method that comprises orally administering the above-described composition pursuant to a prescribed protocol of therapeutic doses on a once daily regimen to a subject that is at risk for or has skin diseases such as acne, eczema, psoriasis, or other related dermatoses.

Generally, the invention is a system comprised by composition of natural compounds for use in dermatological application which is effective for treating skin disorders without the side effects of steroids, antibiotics, anti-fungal, and other treatments. The composition actives include one or more ceramides together with a sulfur compound and a proanthocyanadin, plus adjuvants and excipients.

The composition may optionally include one or more excipients for oral administration. A “physiologically acceptable” excipient is any inert constituent that improves suitability for oral administration such as water, buffered solutions and glucose solutions, among others, stabilizers, preservatives, diluents, emulsifiers or lubricants. In particular, suitable excipients include, but are not limited to, Microcrystalline cellulose, Silicon Dioxide, vegetable magnesium stearate.

### DETAILED DESCRIPTION OF THE PREFERRED EMBODIMENTS

The present invention is a method and composition for treating skin. The composition generally combines a sphingolipid, a sulfur compound, and a proanthocyanidin. The compound preferably includes an adjuvant, preferably a strong anti-oxidant to restore the oxidative balance within the skins subdermal and epidermal environment by the reduction of free radical species, such as ROS, and the reduction of pro-inflammatory processes, such as preventing the release cytokines. In addition, the compound preferably includes excipients designed to facilitate systematic oral administration of a prescribed dosage.

The system targets four synergistic cellular and molecular processes in a combination that is novel in terms of the ability to potentiate the desired activity and outcomes with specificity around particular milligrams per use:
1. System induces reduction of TEWL in order to improve penetration of antibacterial agents and proteolytic enzymes;
2. Increase in NMF in order to improve penetration of antibacterial agents;
3. Modulation of inflammatory pathways: Proteolytic enzymes from Bromelain inhibit the inflammatory process, such as cyclooxygenase (COX) and lipoxygenase (LOX). These enzymes influence the production of pro-inflammatory mediators, including prostaglandins and leukotrienes.
4. Improved delivery of agents to support of antioxidant rich microbiota through the use of ceramide supplementation in the preferred embodiment to a minimum of 20 mg per day per subject.

It is well-known to stay hydrated by drinking water, and that hydrated skin is good. However, saturation of the skin by a stimulator is also a function of hydration. Most stimulants have saturation values where no additional penetration can be achieved without diluting the base. Consequently, the ratio of TWEL to NMF is critical. In the preferred concentrations described herein the combined TEWL and NMF (1-2 above) reach a ratio where the body can improve saturation level of a “stimulator” comprised of antibacterial sulfur and a proanthocyanadin. These processes actively improve the restoration and functionality of the epidermis to greater integrity as a skin barrier in persons with acne, eczema, psoriasis and other similar dermatoses. Additionally, that the system aids in prevention of water loss, maintain thermoregulation, and ward off infection and environmental irritants that may cause disease by aiding repair of damaged stratum corneum of the skin's epidermal cellular architecture using a combination of 3 compounds, and in the preferred embodiment an adjuvant of a protolytic enzyme.

Additionally, the prevention and treatment of infection caused by the over colonization of P. acnes bacteria and/or fungi by leveraging the antibacterial and antifungal properties of a sulfur compound, preferably methysulfonylmethane (MSM) that also has anti-inflammatory and anti-oxidant properties that help to restore the skin's oxidative balance.

Regulating cell proliferation in the epidermis by adding a proteolytic enzyme, preferably derived from bromelain, given that bromelain will work synergistically with a sphingolipid, preferably a ceramide, that is a key component of the enucleated stratified squamous epithelium that constitutes the stratum corneum of the skins epidermal layer.

The specific components of the composition have the following functional effects:

### Ceramides

The epidermis is the most superficial layer of skin and the stratum corneum, its outer most layer, provides a vital permeability barrier against water loss, environmental xenobiotics and harmful microbes. The epidermis is comprised of a lipid rich lamellar matrix that embeds corneocytes generated in its lower layers. These corneocytes are enucleated as they migrate from the more acidic lower epidermal environment to the more alkaline upper epidermal environment during which time they accumulate ceramides due to increased ceramide synthesis.

Ceramides are a family of waxy lipid molecules comprised of sphingosine and fatty acids are crucial for structuring and maintaining skin barrier integrity. They are the major component of the epidermal lamellar matrix that are highly ordered intercellular lamellar structures. Squamous cells of the stratum corneum are comprised of 50% ceramides along with 25% cholesterol, and 15% free fatty acid. Ultra-long chain (C28-C36) ceramides are key components of extracellular lipid lamellae of the stratum corneum. Together with cholesterol and saturated fatty acids, ceramides create water-impermeable skin, a protective organ that prevents excessive exit of water due to evaporation as well as an entry barrier against microorganisms.

Sphingolipid ceramide N-deacylase (SCDase) is an enzyme that is modulated by the pH of its environment, the mechanism by which the synthesis of glycosphingolipids (GSLs) in the epidermis is modulated. SCDase catalyzes reversible reactions in which the amide linkage in glycosphingolipids is hydrolyzed or added to GSL. In a recent invitro study the optimal pH values for the hydrolytic and synthetic activity of SCDase were pH 6.0 and pH 7.5, respectively. Both activities were strongly inhibited by bi-valent zinc (Zn²⁺).

SCDase plays a crucial role in maintaining GSL homeostasis since it catalyzes the reversible hydrolysis/synthesis pathway by removing or adding the amide linkage between the fatty acid and the sphingosine base moieties of the ceramide sphingolipid molecule. SCDase hydrolysis occurs at a greater rate in more acidic environments, such as the lower layers of the epidermis, and at a lesser rate in more alkaline environments, such as occur in the upper layers of the epidermis.

In the more acidic environments, such as the lower layers of the epidermis, SCDase catalyzes the stepwise release of monosaccharide units from the end of the oligosaccharide chains within corneocytes and the lamellar intercellular spaces, leaving just the sphingosine portion of the molecule, contributing to greater production of ceramides. As epidermal corneocytes migrate to the less acidic, more alkaline environments, such as in the upper layers of the epidermis, SCDase activity slows lessening the rate of ceramide synthesis. This ensures the epidermal stratum corneum maintains its integrity as a skin barrier by preventing underproduction of ceramides within enucleated corneocytes and the lamellar intercellular environment, such as is found in eczema, as well as overproduction of the squamous epithelium, as may be found in psoriasis. Additionally, the system provides a particular aid in the delivery and synergetic effects of antibacterial and anti-inflammatory agents to the stratum corneum.

Ceramide and cholesterol decline in the stratum corneum of humans due to aging. In eczema the stratum corneum is hypoplastic and/or disintegrated and in psoriasis the stratum corneum is hyperplastic and exudative. Both dermatoses are characterized by compromised water exit permeability and microbial ingress barrier. The system both improves the ceramide concentration and thus enhances the microenvironment to be more adaptive to immune modulation and penetration of actives that constitute the antibacterial and anti-inflammatory effects synergistically with lipid induction.

### Sulfur

Sulfur has been used in skincare in ancient Indian, Egyptian, Greek and Chinese civilizations. Although it has fallen out of favor in shampoo form, sulfur is still widely used to treat a number of skin conditions in cream, lotion and soaps. The principal dietary sources of sulfur, the amino acids methionine and cysteine, may not always be consumed in adequate amounts to meet sulfur requirements.

Sulfur has a long history of use in skin conditions, and any effect is likely due to the anti-infective properties making it potentially useful in seborrheic dermatitis and acne. Sulfur is an excellent anti-bacterial and antifungal agent and so is most effective in skin conditions associated with underlying infections, such as acne, eczema and psoriasis. It is converted to hydrogen sulfide by bacteria for energy production that quickly becomes toxic to them. Hydrogen sulfide kills a range of organisms, including bacteria, fungi, yeasts, and parasites making it an effective preservative and anti-infective.

Anecdotal evidence online indicates sulfur is exceptionally effective at treating acne. Sulfur soaps, face masks, creams and shampoos can be widely found in alternative health stores, pharmacies, and online retailers in concentrations up to 10%. In acne sulfur helps dry the skin, slows sebum production and its anti-infective properties are effective against P. acnes bacteria. Sulfur compounds also inhibit the growth of Malassezia fungus, which is strongly associated with seborrheic dermatitis.

A 2012 report in the Journal of Medicinal Chemistry determined that sulfonamides were as effective in preventing the fungus' growth than the pharmaceutical ketoconazole 2% prescription strength—the active ingredient found in Nizoral. Sulfur is also mildly keratolytic and can be used as a treatment for psoriasis.

The naturally occurring organosulfur compound, methylsulfonylmethane (MSM), is available as a dietary supplement and has been associated with multiple health benefits. Many medicines contain sulfur preservatives to prevent bacterial growth and improving the shelf-life. These are listed on the ingredient label, with common examples including sulfur dioxide, sodium sulfite, sodium metabisulfite. Each of these has been categorized as ‘generally recognized as safe’, or GRAS, by the US Food and Drug Administration (FDA), and are widely used in tablets and injectables.

### Bromelain

Bromelain is a mixture of proteolytic enzymes extracted from the fruit and stem of the pineapple plant (Ananas comosus). These enzymes belong to a group of proteases, which are enzymes that break down proteins into smaller peptides or amino acids. Bromelain is composed of various proteases.

The proteolytic activity of bromelain makes it useful for a variety of applications, both medicinal and industrial. Bromelain has been studied for its anti-inflammatory effects and it's potential to modulate inflammatory pathways and reduce inflammation associated with various conditions. The activity has been demonstrated to be integrated as functional element to the system herein, including its exertion of fibrinolytic activity which helps breakdown fibrin. This protein involved in blood clotting. The ancillary activity may contribute to improved circulation and reduced redness, promoting a more even skin tone.

### Anti-oxidant

In addition the invention includes the addition of a prothanthocyanadin which possesses a strong anti-oxidant to restore the oxidative balance within the skins subdermal and epidermal environment by the reduction of free radical species. In fact, recently, the application of a proanthocyanadin for the reduction of inflammatory response and its contribution towards a normal endothelial function was elucidated in the recent patent application, patent WO2021/254851. Here both the anti-inflammatory and anti-oxidant activity of proanthocyanadins derived from Maritime French bark have been heavily investigated. ROS, and the reduction of pro-inflammatory processes, such as preventing the release cytokines. are preferably a proanthocyanidin derived from grape seed extract.

Proanthocyanidin is a powerful anti-oxidant that has been widely studied and found in herbs such as burdock root, French tree bark, resveratrol, and grape seed extracts. Grape seed extract, for example, contains oligomeric proanthocyanidins or OPC's which are found to possess and estimated 20 times the antioxidant power of Vitamin C and 50 times that of Vitamin E. Some studies have suggested that the synergistic use of antioxidants provide an important contribution to the treatment of skin disease.

The proanthocyanidin preferably contains 70% OPCs and can be extracted from pine bark, peanut skin, grape seed, witch hazel bark, etc., yielding good proanthocyanidin coverage to reduce oxidative stress and help protect the body against free radicals.

The invention also utilizes the addition of a proteolytic enzyme, Bromelain. Bromelain is a mixture of proteolytic enzymes extracted from the fruit and stem of the pineapple plant (Ananas comosus). These enzymes belong to a group of proteases, which are enzymes that break down proteins into smaller peptides or amino acids. The benefit of the enzyme in the stated system is to enhance the cellular process of mediating the immune response, as Bromelain is known to modulate the production of various inflammatory mediators, including cytokines and chemokines. These molecules play crucial roles in the initiation and propagation of inflammatory responses. Additionally, the use of bromelain has been reported to reduce edema or swelling associated with inflammatory responses. This effect is partly due to its ability to modulate the production of inflammatory mediators and control the permeability of blood vessels, helping to maintain the integrity and function of the skin barrier. Moreover, it compounds the inhibiting factor of bacterial adhesion to molecules. Inflammatory responses involve the adhesion of immune cells to blood vessel walls. Bromelain has been suggested to interfere with the expression of adhesion molecules, reducing the recruitment of immune cells to inflamed tissues. A lesser, but documented effect of bromelain is its exhibition of antioxidant properties, helping to scavenge free radicals. Free radicals contribute to oxidative stress, which can exacerbate inflammatory conditions. By reducing oxidative stress, bromelain contributes to its anti-inflammatory effects.

The study of phytochemicals, minerals and vitamins have contributed greatly to the field of biochemical and molecular biology

Through the iterative references that are disclosed in the literature, in addition to various supplements and nutraceuticals marketed to consumers today, none of these disclose or suggest the combination use of a sphingolipid, sulfur compound, and proanthocyanadin compound with a proteolytic enzyme adjuvant as dermatological agents to be used for managing specific dermatological conditions. What is needed is a method and composition to improve the human skin condition, while at the same time not disrupting the body's own microbiome or having other unwanted side effects.

Indeed, such an approach would not only contribute to alleviating skin disease but improve the overall life of the recipient. The present invention provides such a composition that acts by facilitating corrections to the physical condition of the skin that has been impacted by the disease. The result is a reduction in ROS, inhibiting cytokine release and thereby reducing suffering of the patient while also improving recovery time. Equally important is the longevity of sustained recovery with use. The condition of the skin tissues results in improved barrier performance by the addition of moisture retention and toxic flushing of the organ by the hydration and rehydration process.

The composition may optionally include one or more excipients for oral administration. A “physiologically acceptable” excipient is any inert constituent that improves suitability for oral administration such as water, buffered solutions and glucose solutions, among others, stabilizers, preservatives, diluents, emulsifiers or lubricants. In particular, suitable excipients include, but are not limited to, Microcrystalline cellulose, Silicon Dioxide, vegetable magnesium sterate.

A preferred embodiment of the above-described therapeutic composition may comprise the following quantities of constituents in conformance with the following Table:

| Constituents | Preferred Amount | Acceptable Range |
|---|---|---|
| Phytoceramide | 40 mg | +/−50% |
| Sulfur | 150 mg | +/−50% |

MSM (methylsulfonylmethane), also known as
dimethyl sulfone (DMSO2) and methyl sulfone

| | | |
|---|---|---|
| Grape Seed Extract | 100 mg | +/−40% |

Oligomeric proanthocyanidins (OPC), e.g., plant-based dimer
and trimer polymerizations of catechins extracted from
pine bark, peanut skin, grape seed, witch hazel bark, etc.

| | | |
|---|---|---|
| Adjuvant | | |
| Bromelain | 150 mg | +/−40% |

### Excipients

Microcrystalline Cellulose
Silicon Dioxide
Vegetable Magnesium Stearate

The method of administration comprises oral delivery of a therapeutic dose of the above-described composition in single tablet or capsular form taken once or twice daily by a person of average size. The permissible ranges eliminate possible toxicity.

A clinical investigation was conducted using a placebo-controlled double-blind ingestion study followed by microscopic three-dimensional imaging analysis in dry skin in 33 subjects who always tended to have rough skin due to dryness. Results show a significant improvement of dryness and itching of the skin, increase in water content, and marked improvement to smoothness, exfoliation, and texture of the skin. The above findings evidence the fact that long-term ingestion of an oral supplement as described is effective in moisture-retention and maintaining smoothness, health and aesthetics of the skin.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A composition for the prevention and treatment of skin conditions, comprising
a sphingolipid; and
proanthocyanadin.

2. The composition for the prevention and treatment of skin conditions according to claim 1, further comprising sulfur.

3. The composition for the prevention and treatment of skin conditions according to claim 1, wherein said sphingolipid is a ceramide.

4. The composition for the prevention and treatment of skin conditions according to claim 3, wherein said ceramide comprises a phytoceramide.

5. The composition for the prevention and treatment of skin conditions according to claim 4, wherein said phytoceramide comprises an amount by weight within a range of from 20 mg to 60 mg.

6. The composition for the prevention and treatment of skin conditions according to claim 5, wherein said phytoceramide comprises approximately 40 mg by weight.

7. The composition for the prevention and treatment of skin conditions according to claim 1, further comprising one or more adjuvants.

8. The composition for the prevention and treatment of skin conditions according to claim 1, further comprising at least one proteolytic enzyme adjuvant.

9. The composition for the prevention and treatment of skin conditions according to claim 8, wherein said at least one adjuvant comprises bromelain.

10. The composition for the prevention and treatment of skin conditions according to claim 9, wherein said bromelain comprises an amount by weight within a range of from 90 mg to 150 mg.

11. The composition for the prevention and treatment of skin conditions according to claim 10, wherein said bromelain comprises approximately 150 mg by weight.

12. The composition for the prevention and treatment of skin conditions according to claim 1, further comprising one or more excipients for oral administration.

13. The composition for the prevention and treatment of skin conditions according to claim 12, wherein said excipients for oral administration comprise any one or more from among the group consisting of Microcrystalline cellulose, Silicon Dioxide, and vegetable magnesium sterate.

14. A composition for the prevention and treatment of skin conditions, comprising
a sphingolipid; and
sulfur.

15. The composition for the prevention and treatment of skin conditions according to claim 14, further comprising bromelain.

16. The composition for the prevention and treatment of skin conditions according to claim 14, wherein said sphingolipid is a ceramide.

17. The composition for the prevention and treatment of skin conditions according to claim 16, wherein said ceramide comprises a phytoceramide.

18. The composition for the prevention and treatment of skin conditions according to claim 17, wherein said phytoceramide comprises an amount by weight within a range of from 20 mg to 60 mg.

19. The composition for the prevention and treatment of skin conditions according to claim 18, wherein said phytoceramide comprises approximately 40 mg by weight.

20. The composition for the prevention and treatment of skin conditions according to claim 14, further comprising one or more adjuvants.

21. The composition for the prevention and treatment of skin conditions according to claim 14, further comprising at least one anti-oxidant adjuvant.

22. The composition for the prevention and treatment of skin conditions according to claim 21, wherein said at least one adjuvant comprises bromelain.

23. The composition for the prevention and treatment of skin conditions according to claim 22, wherein said proanthocyanidin comprises an amount by weight within a range of from 30 mg to 70 mg.

24. The composition for the prevention and treatment of skin conditions according to claim 23, wherein said proanthocyanidin comprises approximately 50 mg by weight.

25. The composition for the prevention and treatment of skin conditions according to claim 14, further comprising one or more excipients for oral administration.

26. The composition for the prevention and treatment of skin conditions according to claim 25, wherein said excipients for oral administration comprise any one or more from among the group consisting of Microcrystalline cellulose, Silicon Dioxide, vegetable magnesium sterate.

27. A composition for treating skin disorders comprising:
at least one ceramide;
at least one sulfur compound
at least one proanthocyanadin compound
at least one proteolytic enzyme adjuvant and
a plurality of excipients.

28. The composition according to claim 27, wherein said at least one ceramide comprises a rice-derived phytoceramide.

29. The composition according to claim 27, wherein said at least one sulfur compound comprises methylsulfonylmethane (MSM)

30. The composition according to claim 27, wherein said at least one proanthocyanadin compound comprises grape seed extract

31. The composition according to claim 27, wherein at least one adjuvant comprises a bromelain grape seed derive proteolytic enzymes

32. A method of administration of the composition of claim 1 comprising a step of orally ingesting said composition in tablet or capsular form once per day.
